(19) United States
(12) Patent Application Publication
(10) Pub. No.: US 2026/0102564 A1
(43) Pub. Date: Apr. 16, 2026

(54) SYSTEMS, METHODS, AND DEVICES FOR TARGET CONTROLLED INFUSION

(71) Applicant: CAREFUSION 303, INC., San Diego, CA (US)

(72) Inventors: Richard BAILEY, Basingstoke (GB); Jan Henning AUSTNES, Snaroeya (NO)

(21) Appl. No.: 19/358,153

(22) Filed: Oct. 14, 2025

Related U.S. Application Data
(60) Provisional application No. 63/707,120, filed on Oct. 14, 2024.

(51) Int. Cl. A61M 5/172; G16H 20/17; G16H 40/67

(52) U.S. Cl. CPC A61M 5/1723; G16H 20/17; G16H 40/67; A61M 2205/3303; A61M 2205/505; A61M 2205/584

(57) ABSTRACT

A system for programming infusion devices for target controlled infusion is disclosed. The system includes a docking station and one or more processors configured to determine a first infusion device docked with a docking station and accept user input of one or more of patient-specific parameters, drug-related parameters, and a desired target concentration of a therapeutic agent in a workflow on the first infusion device for programming a target controlled infusion (TCI) therapy. After receiving a particular set of parameters, the set of parameters can be copied to the second infusion device to pre-complete and bypass a number of workflow steps of a second workflow of another infusion device. When the second workflow is accessed, the number of workflow steps of the second graphical interface workflow are pre-completed and bypassed.

### CROSS-REFERENCE TO RELATED APPLICATIONS

This application claims the priority from U.S. Provisional Application No. 63/707,120, filed on Oct. 14, 2024, the entirety of which is incorporated herein by reference for all purposes.

### FIELD

The innovative aspects described herein generally relate to systems and methods for safely programming medical devices, and more particularly to programming infusion devices for target controlled infusion (TCI) of therapeutic agents.

### BACKGROUND

Various medical devices and instruments typically found in a healthcare institution are used to provide medication, monitor patient condition, and assist in the diagnosis and treatment of disease. Common to many of these devices is the need to input therapeutic or patient-related parameters that are used to identify the device, configure operational settings, or provide data to a computer-controlled system, which may be ward-based or institution wide, to monitor and record diagnoses and treatment related to a particular patient.

One example of a medical device where the entry of numerous parameters is required is an infusion device or infusion pump, which is used to administer medication to a patient. An infusion pump may deliver fluids or drugs in small, precisely measured doses at defined intervals, or continuously at controlled rates. Such devices generally include user interfaces, such as keypads or touchscreens, through which a clinician programs infusion parameters such as drug type, syringe size, infusion rate, or duration of infusion. The device may further include a display for viewing programmed parameters and real-time infusion status while an infusion is in progress.

As the range of therapeutic drugs and clinical techniques has expanded, so too has the demand for greater sophistication and precision in drug delivery. However, increasing the complexity of infusion control, including the number of parameters utilized in infusions, can make devices more difficult to operate, introducing cognitive and data entry burden and the potential for user error. Manual input of multiple parameters can be time-consuming and also prone to error, particularly in critical care settings. Consequently, achieving advanced drug delivery capabilities while maintaining ease of use and safety has become a design challenge for infusion pump manufacturers.

To address these challenges, automation of therapy programming and delivery has been pursued. One such approach is known as target controlled infusion (TCI). TCI includes a computer-assisted method of drug delivery that automates the infusion process based on a defined target drug concentration in the patient (e.g., the concentration desired in a patient's blood plasma or at a specific site of drug action). Rather than the clinician setting an infusion rate, the clinician specifies a target concentration, and the TCI system calculates and adjusts the infusion rate dynamically to achieve and maintain that concentration.

TCI systems have been shown to enhance safety, efficiency, and precision in drug administration, particularly during anesthesia and sedation. In comparison to manually controlled infusions, TCI can achieve more stable plasma and effect-site concentrations with reduced workload for anesthetists and other healthcare providers. Safety features may include continuous monitoring of infusion progress, automated alarms or stop functions in response to abnormal conditions, and safeguards against exceeding safe dosing limits.

In clinical practice, TCI is particularly valuable in surgical and critical care settings, where precise titration of anesthetic or sedative agents is essential. For example, TCI systems are commonly used to deliver total intravenous anesthesia (TIVA) by automatically adjusting the infusion of agents such as propofol or remifentanil to maintain the desired depth of anesthesia.

Despite these advances, existing TCI systems still present limitations that can affect safety, usability, and reliability. Programming errors may arise from manual data entry, incorrect selection of pharmacokinetic models, or incomplete synchronization between the infusion device and external data systems. As such, there remains a need for improved TCI programming and control mechanisms that enhance automation while ensuring safety, reducing clinician workload, and minimizing the potential for human or system error.

### SUMMARY

The systems and methods described herein address the foregoing limitations by providing techniques for safely programming and operating infusion devices configured for target controlled infusion (TCI). In various implementations, the subject technology enables infusion devices to automatically determine, verify, or adjust infusion parameters associated with a pharmacokinetic or pharmacodynamic model while maintaining safety, accuracy, and ease of use.

According to some implementations, a machine implemented method for programming infusion devices for target controlled infusion, comprises: determining a first infusion device is docked with a docking station configured to receive a plurality of infusion devices; receiving, via a first graphical interface workflow displayed on the first infusion device, user input of one or more of patient-specific parameters, drug-related parameters, and a desired target concentration of a therapeutic agent for programming and initiation of a first target controlled infusion (TCI) therapy provided by the first infusion device; determining when a second infusion device becomes docked with the docking station; after receiving the patient-specific parameters, the drug-related parameters, or the desired target concentration, and responsive to determining that the first and second infusion devices are simultaneously docked with the docking station, copying a set of parameters to the second infusion device to pre-complete a number of workflow steps of a second graphical interface workflow for programming and initiating a second TCI therapy provided by the second infusion device; and wherein, after copying the set of parameters to the second infusion device and the second graphical interface workflow is displayed on a display screen of the second infusion device, the number of workflow steps of the second graphical interface workflow are pre-completed and bypassed such that user input is not required for completion of the number of workflow steps of the second graphical interface workflow to program and initiate the second TCI therapy, wherein the number of workflow steps of the second graphical interface workflow would require user input had the set of parameters not been copied.

The machine implemented method may further comprise receiving an indication that the second graphical interface workflow was completed on the second infusion device for programming and initiation of a second TCI therapy provided by the second infusion device; and causing the second infusion device to initiate the second TCI therapy based on the set of parameters copied to the second infusion device. Other aspects include corresponding systems, apparatus, and computer program products for implementation of the corresponding method and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description below, in conjunction with the following drawings. Like reference numerals refer to corresponding parts throughout the figures and description.

FIG. 1 depicts an example infusion device, according to various aspects of the subject technology.

FIG. 2A depicts an example of an institutional patient care system of a healthcare organization, according to aspects of the subject technology.

FIG. 2B depicts a number of syringe pumps configured on a docking station, according to various aspects of the subject technology.

FIG. 3 depicts respective workflow processes implemented by respective infusion devices to configure the infusion devices to perform a target controlled infusion (TCI), according to aspects of the subject technology.

FIG. 4 depicts an example medication safety software (MSW) editor for generation of a therapy setup library for programming infusion devices for target controlled infusion, according to aspects of the subject technology.

FIG. 5 depicts an example process for programming infusion devices for target controlled infusion, according to aspects of the subject technology.

FIG. 6 is a conceptual diagram illustrating an example electronic system for preconditioning a syringe for bolus delivery, according to aspects of the subject technology.

### DETAILED DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

A system and method for programming infusion devices for target controlled infusion is disclosed. In some implementations, the disclosed system includes a processor configured to receive one or more patient-specific parameters (e.g., age, weight, sex, height, health condition), drug-related parameters (e.g., concentration, pharmacokinetic model, or therapeutic range), and a desired target concentration of the therapeutic agent. The processor may execute a control algorithm that uses this data to determine infusion control commands for control of an infusion device to safely achieve and maintain target concentrations of drugs in the patient. The system may further include a model management module configured to select or adapt pharmacokinetic and pharmacodynamic models in accordance with patient characteristics, drug selection, or institutional protocol. This enables dynamic adjustment of model parameters or infusion profiles in response to patient monitoring data or clinician input, thereby improving personalization and responsiveness of the TCI process.

According to various implementations described herein, TCI drug models and algorithms may be fixed and made available to numerous different pump manufacturers, while the setup of such models and algorithms may remain distinguishable between respective infusion devices.

The subject technology provides TCI workflows across infusion devices that include integration of drug models into each administered therapy/infusion. The workflows enable the user to set up TCI quickly and safely for multiple infusion devices and provide operational monitoring and adjustment of the infusion device's performance.

The subject technology provides a mechanism for copying parameters from one workflow to another when the respective infusion devices are operably connected, for example, by way of being docked in a docking station. Existing systems for copying parameters have failed to provide an intuitive, efficient, and safe transfer required to avert human error, and have not been implemented in the context of a workflow system. The subject technology provides for the publishing of data stored on one infusion device, within the context of the workflow steps, to other infusion devices providing TCI to the same patient. Accordingly, the same workflow steps on the other devices may be automatically populated and bypassed, thereby eliminating unnecessary redundancies of repeating the workflow steps, particularly when all the infusion devices are being setup to provide therapy to the same patient. In this regard, human errors that may result from repeated data entry may be avoided in critical scenarios.

Moreover, a medication safety software (MSW) editor enables the configuration and set up of TCI/total intravenous anesthesia (TIVA), further reducing workflow steps when setting up infusion devices for TCI. For example, if only one TCI drug is selected in the MSW editor the clinical workflow steps of the infusion device may be reduced compared to if two drugs were selected in the drug library editor.

In other aspects, it has been suggested that clinicians setting up two infusion devices may erroneously program one device with one or more parameters of the other device. This becomes problematic in situations where two different drugs are infused by the devices. For example, during anesthesia, a clinician may set up a Propofol TCI infusion (for sedation) and Remifentanil infusion (for pain management) and put the pumps on hold. The drugs are made up while the infusion devices are on hold, and the clinician may erroneously place the propofol drug into the infusion device that was previously setup for remifentanil and the remifentanil drug into the infusion device that is set up for propofol. Initiating the infusions under these conditions may result in severe and avoidable consequences for the patient. Systems employing RFID enabled medication containers/syringes are expensive and prone to error.

The disclosed system adjusts the displays of the respective infusion devices based on the drugs identified in the setup of each device. When an infusion device is set up for propofol and then placed in standby/on hold (so that the drug can be loaded), it is configured to clearly indicate that the infusion device is using propofol by way of displaying readily perceivable indicators. In some implementations, the user interface of the infusion device is changed to a predetermined color (e.g., yellow) associated with the particular drug (propofol).

Moreover, the disclosed system may further provide real-time feedback control by acquiring physiological or pharmacological measurements, such as heart rate, blood pressure, or drug concentration estimates, and adjust infusion parameters accordingly. This closed-loop or semi-closed-loop operation enhances dosing precision and ensures that drug concentrations remain within safe therapeutic ranges.

The subject technology described herein thus provides improved safety, automation, and interoperability for target controlled infusion devices. By combining patient-specific modeling, automated data verification, and networked communication capabilities, the disclosed systems facilitate accurate and reliable TCI operation while reducing clinician workload and minimizing opportunities for human error.

While the methods and systems disclosed herein are described with regard to syringe pumps, the subject technology is applicable to all types of infusion pumps, including large volume pumps.

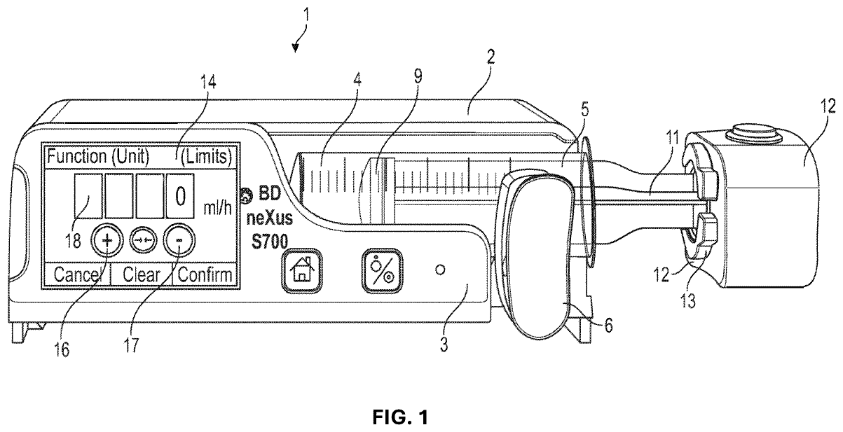

FIG. 1 depicts an example infusion device 1, according to various aspects of the subject technology. Infusion device 1 includes a housing 2 having a syringe cradle 3 therein which is of appropriate size for receiving a syringe 4, in particular the syringe barrel 5 thereof.

While the depicted infusion device 1 includes a syringe pump, other types of infusion devices are also contemplated. For example, infusion device 1 may include a peristaltic pump that drives a fluid through an intravenous (IV) tube.

In the example shown, there is a clip 6 provided to support the syringe barrel 5 in the syringe cradle 3 and a groove which receives the flange 7 of the syringe barrel 5. The prior art syringe pump 1 as shown is in a state either before or during infusion where the syringe plunger 8 is extended out of the syringe barrel 5. The syringe plunger 8 terminates with a syringe piston 9 at one end, which forces fluid from the syringe 4 and a syringe flange 10 at an opposing end, connected by the syringe plunger stem 11. The syringe flange 10 is pushed via the driver head 12 when in use, which forces the syringe piston 9 through the syringe barrel 5 thereby forcing liquid through the end of the syringe 4. The syringe flange 10 may additionally be supported or clamped by one or more retaining arms 13.

As well as operating buttons or switches, which the operator uses to activate and program the syringe pump 1, there is a display screen 14. The display screen 14 may be a simple LCD (liquid crystal display) having a small number of segments, for example seven segments in a figure-of-eight configuration per character, adapted to display a small number of alphanumeric characters. The display may be monochromatic, for example, it might only display red, green or grey/black characters. Alternatively, the display 14 might be a more complicated liquid crystal display capable of displaying more characters or more complicated characters. The LCD may be backlit, for example, using light emitting diodes (LEDs). In some implementations, the infusion pump may include a TFT LCD. A TFT is a thin-film transistor-based LCD technology. In some implementations, the display screen 14 is also a touchscreen such as a capacitive or resistive touchscreen. Such a display screen 14 may be used to present one or more of the user interfaces or workflow steps shown in Exhibit A.

When programming an infusion device, such as part of an example workflow in Exhibit A, the user must input the type of syringe 4 being fitted to the pump. The pump stores in an internal memory a database of known syringe types containing information such as syringe diameter and stroke. The infusion pump firmware calculates the position of the syringe plunger and syringe piston based on movement of the syringe driver head and the type and size of the syringe. This allows the machine to display the calculation of volume infused, time elapsed, volume remaining and time remaining. As infusion continues and the driver head moves, these calculations can be updated, and the displayed information changed.

Infusion device 1 may be provided with a scrolling system comprising an “up” scroll key and a “down” scroll key which are operable to increase or decrease pumping parameters, such as the mass flow rate setting shown on a display, or the VTBI (volume to be infused) setting shown on the display. In some cases, each scroll key may be labeled with upward 16 and downward 17 symbols or chevrons. Pressing either of the keys causes the display to scroll numerically upwardly or downwardly.

For example, assuming that the display shows “6”, pressing the chevron UP key causes the display to scroll up by one unit at a time to show “7”, “8”, “9”, “10”, “11”, and so on. The display may be caused to scroll up in one unit increments (e.g. 1 ml) until “20” and then may automatically switch to scroll up further in ten unit increments until it reaches, for example, a display of “200”, where after continued pressing of the chevron UP key may cause the display to increase in hundred unit increments to show “300”, “400”, and so on. When the display reaches a certain number, such as “700”, pressing the chevron UP key may cause the display to move again in single increments, such as “701”, “702”, “703”, and so on. The same or similar scheme may be performed when decrementing using the DOWN key 17 (in reverse).

Display screen 14 may be used for inputting parameters, or to enter data for use by the device 1 in monitoring and recording the status or condition of a patient or a course of treatment. For example, a processor of pump 1 may communicate infusion parameters input by a user to a pump controller (not shown). Display panel 14 may be configured to show four character or digit fields 18. Of course, the display may be configured to show more than four fields, so as to accommodate inputting numerical values greater than “9999” or less than “−9999”, or numerical values having an accuracy greater than five or more significant digits, such as “1.0001”. As will be described further, display panel 14 may further include a focus indicator designating a field 18 selected for input. The focus indicator is useful in that it identifies which digit or character field 18 displayed on display 14 that is active, that is, can be changed using the controls of the device or by touching a displayed character field 18. The term character field and digital field are used synonymously herein.

The UP key 16 and DOWN key 17 may be touch elements on display screen 14, as depicted, or may be physical switches, such as bubble-style or membrane switches. Such keys are displayed as needed depending on the configuration and programming of the infusion pump or medical device. Other types of keys or icons, apart from the UP and DOWN keys illustrated in FIG. 1 may be used in order to change the value shown on the display 14. For example, track balls, joysticks, touch pads, and scroll wheels may be used for scrolling the displayed value either upwards or downwards.

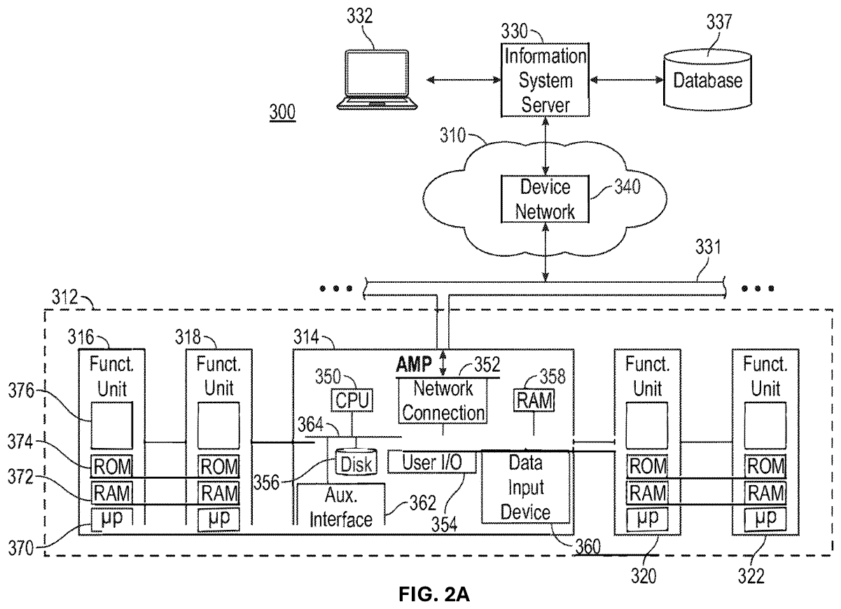

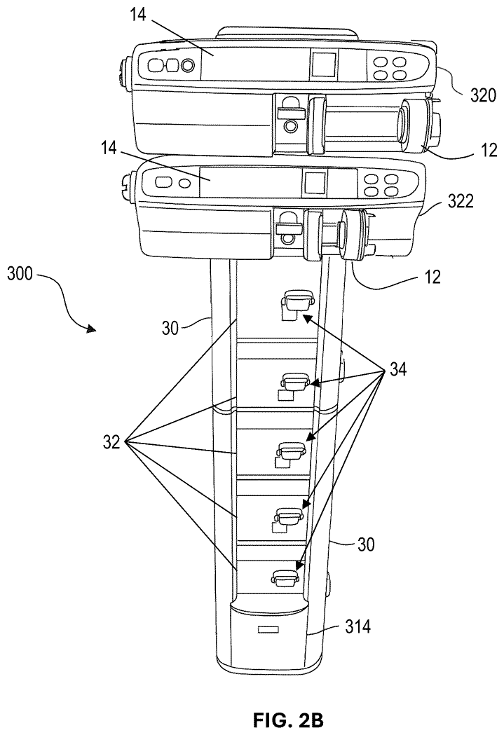

FIG. 2A depicts an example of an institutional patient care system 300 of a healthcare organization, according to aspects of the subject technology. In FIG. 2A, a patient care device (or “medical device” generally) 312 is connected to a hospital network 310. The term patient care device (or “PCD”) may be used interchangeably with the term patient care unit (or “PCU”), either which may include various ancillary medical devices such as an infusion pump, a vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. According to some implementations, patient care device 312 may be representative of the previously described infusion device 1.

Patient care device 312 may be connected to an internal healthcare network 310 by a transmission channel 331. Transmission channel 331 is any wired or wireless transmission channel, for example an 802.11 wireless local area network (LAN). In some implementations, network 310 also includes computer systems located in various departments throughout a hospital. For example, network 310 of FIG. 2A optionally includes computer systems associated with an admissions department, a billing department, a biomedical engineering department, a clinical laboratory, a central supply department, one or more unit station computers and/or a medical decision support system. As described further below, network 310 may include discrete subnetworks. In the depicted example, network 310 includes a device network 340 by which patient care devices 312 (and other devices) communicate in accordance with normal operations.

Additionally, institutional patient care system 100 may incorporate a separate information system server 330, the function of which will be described in more detail below. Moreover, although the information system server 330 is shown as a separate server, the functions and programming of the information system server 330 may be incorporated into another computer, if such is desired by engineers designing the institution's information system. Institutional patient care system 300 may further include one or multiple device terminals 332 for connecting and communicating with information system server 330. Device terminals 332 may include personal computers, personal data assistances, mobile devices such as laptops, tablet computers, augmented reality devices, or smartphones, configured with software for communications with information system server 330 via network 310. The server 330 or terminal 332 may be configured to present the medication safety workstation features shown in Exhibit A.

Patient care device 312 includes a system for providing patient care, such as that described in Eggers et al., which is incorporated herein by reference for that purpose. Patient care device 312 may include or incorporate pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care device 312 comprises a control module 314, also referred to as interface unit 314, connected to one or more functional modules 316, 318, 320, 322. Interface unit 314 includes a central processing unit (CPU) 350 connected to a memory, for example, random access memory (RAM) 358, and one or more interface devices such as user interface device 354, a coded data input device 360, a network connection 352, and an auxiliary interface 362 for communicating with additional modules or devices. Interface unit 314 also, although not necessarily, includes a main non-volatile storage unit 356, such as a hard disk drive or non-volatile flash memory, for storing software and data and one or more internal buses 364 for interconnecting the aforementioned elements.

In various implementations, user interface device 354 is a touch screen for displaying information to a user and allowing a user to input information by touching defined areas of the screen. Additionally, or in the alternative, user interface device 354 could include any means for displaying and inputting information, such as a monitor, a printer, a keyboard, softkeys, a mouse, a track ball and/or a light pen. Data input device 360 may be a bar code reader capable of scanning and interpreting data printed in bar coded format. Additionally or in the alternative, data input device 360 can be any device for entering coded data into a computer, such as a device(s) for reading a magnetic strips, radio-frequency identification (RFID) devices whereby digital data encoded in RFID tags or smart labels (defined below) are captured by the reader 360 via radio waves, PCMCIA smart cards, radio frequency cards, memory sticks, CDs, DVDs, or any other analog or digital storage media. Other examples of data input device 360 include a voice activation or recognition device or a portable personal data assistant (PDA). Depending upon the types of interface devices used, user interface device 354 and data input device 360 may be the same device. Although data input device 360 is shown in FIG. 2A to be disposed within interface unit 314, it is recognized that data input device 360 may be integral within a pharmacy system or located externally and communicating with pharmacy system through an RS-232 serial interface or any other appropriate communication means. Auxiliary interface 362 may be an RS-232 communications interface, however any other means for communicating with a peripheral device such as a printer, patient monitor, infusion pump or other medical device may be used without departing from the subject technology. Additionally, data input device 360 may be a separate functional module, such as modules 316, 318, 320 and 322, and configured to communicate with controller 314, or any other system on the network, using suitable programming and communication protocols.

Network connection 352 may be a wired or wireless connection, such as by Ethernet, WiFi, BLUETOOTH, an integrated services digital network (ISDN) connection, a digital subscriber line (DSL) modem or a cable modem. Any direct or indirect network connection may be used, including, but not limited to a telephone modem, an MIB system, an RS232 interface, an auxiliary interface, an optical link, an infrared link, a radio frequency link, a microwave link or a WLANS connection or other wireless connection.

Functional modules 316, 318, 320, 322 are any devices for providing care to a patient or for monitoring patient condition. As shown in FIG. 2A, at least one of functional modules 316, 318, 320, 322 may be an infusion pump module such as an intravenous infusion pump for delivering medication or other fluid to a patient. For the purposes of this discussion, functional module 316 is an infusion pump module. Each of functional modules 318, 320, 322 may be any patient treatment or monitoring device including, but not limited to, an infusion pump, a syringe pump, a PCA pump, an epidural pump, an enteral pump, a blood pressure monitor, a pulse oximeter, an EKG monitor, an EEG monitor, a heart rate monitor or an intracranial pressure monitor or the like. Functional module 318, 320 and/or 322 may be a printer, scanner, bar code reader or any other peripheral input, output or input/output device.

Each functional module 316, 318, 320, 322 communicates directly or indirectly with interface unit 314, with interface unit 314 providing overall monitoring and control of device 312. Functional modules 316, 318, 320, 322 may be connected physically and electronically in serial fashion to one or both ends of interface unit 314 as shown in FIG. 2A. However, it is recognized that there are other means for connecting functional modules with the interface unit that may be utilized without departing from the subject technology. It will also be appreciated that devices such as pumps or patient monitoring devices that provide sufficient programmability and connectivity may be capable of operating as stand-alone devices and may communicate directly with the network without connected through a separate interface unit or control unit 314. As described above, additional medical devices or peripheral devices may be connected to patient care device 312 through one or more auxiliary interfaces 362.

Each functional module 316, 318, 320, 322 may include module-specific components 376, a microprocessor 370, a volatile memory 372 and a nonvolatile memory 374 for storing information. It should be noted that while four functional modules are shown in FIG. 2A, any number of devices may be connected directly or indirectly to central controller 314. The number and type of functional modules described herein are intended to be illustrative, and in no way limit the scope of the subject technology. Module-specific components 376 include any components necessary for operation of a particular module, such as a pumping mechanism for infusion pump module 316.

While each functional module may be capable of a least some level of independent operation, interface unit 314 monitors and controls overall operation of device 312. For example, as will be described in more detail below, interface unit 314 provides programming instructions to the functional modules 316, 318, 320, 322 and monitors the status of each module.

Patient care device 312 is capable of operating in several different modes, or personalities, with each personality defined by a configuration database. The configuration database may be a database 356 internal to patient care device, or an external database 337. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics or psychological characteristics. As used herein, patient-specific information also includes care provider information (e.g., physician identification) or a patient care device's 312 location in the hospital or hospital computer network. Patient care information may be entered through interface device 354, or the data input device 360, or auxiliary interface 362, and may originate from anywhere in network 310, such as, for example, from a pharmacy server, admissions server, laboratory server, and the like.

Medical devices incorporating aspects of the subject technology may be equipped with a Network Interface Module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care device 312 and network 310 may communicate via automated interaction, manual interaction or a combination of both automated and manual interaction. Automated interaction may be continuous or intermittent and may occur through direct network connection 352 (as shown in FIG. 2A), or through RS232 links, MIB systems, RF links such as BLUETOOTH, IR links, WLANS, digital cable systems, telephone modems or other wired or wireless communication means. Manual interaction between patient care device 312 and network 310 involves physically transferring, intermittently or periodically, data between systems using, for example, user interface device 354, coded data input device 360, bar codes, computer disks, portable data assistants, memory cards, or any other media for storing data. The communication means in various aspects is bidirectional with access to data from as many points of the distributed data sources as possible. Decision-making can occur at a variety of places within network 310. For example, and not by way of limitation, decisions can be made in a remote data server, a hospital department or unit stations, or within patient care device 312 itself.

All direct communications with medical devices operating on a network in accordance with the subject technology may be performed through information system server 30, known as the remote data server (RDS). In accordance with aspects of the subject technology, network interface modules incorporated into medical devices such as, for example, infusion pumps or vital signs measurement devices, ignore all network traffic that does not originate from an authenticated RDS. The primary responsibilities of the RDS of the subject technology are to track the location and status of all networked medical devices, and maintain open communication.

With further reference to FIG. 2A, an infusion device, as used herein, may be a patient care device 312, interface control unit 314, or a module 316, 318, 320, 322. According to various implementations, the infusion device includes a pump and a control unit. The control unit 314 is configured to provide, using the pump, an intravenous infusion of a medication to a current patient, and display on a display screen, while the infusion is being provided by the pump, a representation of a status of the intravenous infusion. The representation of the status may include, for example, a single colored light emanating from an LED affixed to the control unit, pump, or infusion device generally, or may be an element displayed on the display screen. In some implementations, the colored light bay be a backlight or lighted outline around content in a display screen.

Control unit 314 is configured to send, during the infusion, to a remote records system 330, infusion information including a patient identifier and order identifier for the infusion currently being provided by the pump. For example, a caregiver may input that a particular patient is receiving a certain dose of acyclovir, and the patient ID, drug ID, and dosage information may be sent to system 330 along with the device ID of the infusion device. Records system 330 may then send to control unit 314 (for which control unit 314 is configured to then receive), a confirmation of the infusion information and any limits for the medication and or the patient based on the information received, as described previously.

FIG. 2B depicts a number of syringe pumps 1 configured on a docking station 300, according to various aspects of the subject technology. In this regard, the syringe pumps may be mounted together for example on a mounting assembly such as a “I” piece 30 or a pole, together with the optional control unit 314, to which all of the pumps 1 are connected. This means that a number of infusions can be carried out from separate syringe pumps 1, either simultaneously or sequentially, optionally under the control of the control module 314. Similar to the patient care device 312 configuration of FIG. 2A, the depicted docking station 300 provides a common mechanical, electrical, and communication interface through which multiple infusion modules 316, 318, 320, 322 can be mounted, powered, and networked in a clinical environment. For the purposes of this disclosure, the configurations of patient care device 312 and docking station 300 may be referred to interchangeably.

As in the depicted example, the docking station 300 may include an elongated base housing 30 defining a series of docking positions 32 including interface bays or receptacles for docking respective syringe pumps 1, as respective functional units 320, 322. Each docking position 32 may include, for example, a guide rail or latch for mechanical retention and an electrical connector 34 for power and/or communication. In some implementations, each connector 34 may include a communication interface configured to mate with a corresponding connector and engagement feature on a compatible syringe pump module 1. When a syringe pump is docked, the connector engages a power bus and data bus within the station, thereby supplying regulated power and enabling bidirectional communication with a control module 314 or connected hospital network. In some implementations, communication between docked infusion devices may be by way of direct cable connections (e.g., USB, Ethernet, and the liked) or wireless connections (e.g., Bluetooth, WiFi, mesh network, and the like) between the docked infusion devices.

The docking station may incorporate an internal power supply or derive power from an external source, distributing electrical energy to each connected pump through independent circuits. The control unit 314 within the docking station 300 may receive and aggregate data traffic from all connected pumps and provide further communication to a network interface 352 (e.g., Ethernet, USB, or wireless interface) for integration with a hospital information system 330 over a network 310, 340. Through this connection, the docking station enables consolidated data logging, remote monitoring, or coordinated alarm management for multiple infusion devices.

In a typical clinical environment, the docking station 300 may be positioned at a patient's bedside, in an operating room, or proximate an anesthesia workstation to consolidate multiple syringe or large volume infusion pumps delivering medications to the same patient.

Variations of the docking station 300 may differ in form factor or capability. In some implementations, each docking station may function purely as a passive backplane providing power, while communication routing is achieved via cabling between individual infusion devices. Some implementations may incorporate processors, touchscreens, or networking interfaces for centralized pump control. The number of docking positions may vary, and the mechanical interface may accept different pump types such as syringe, large volume pumps, or peristaltic modules.

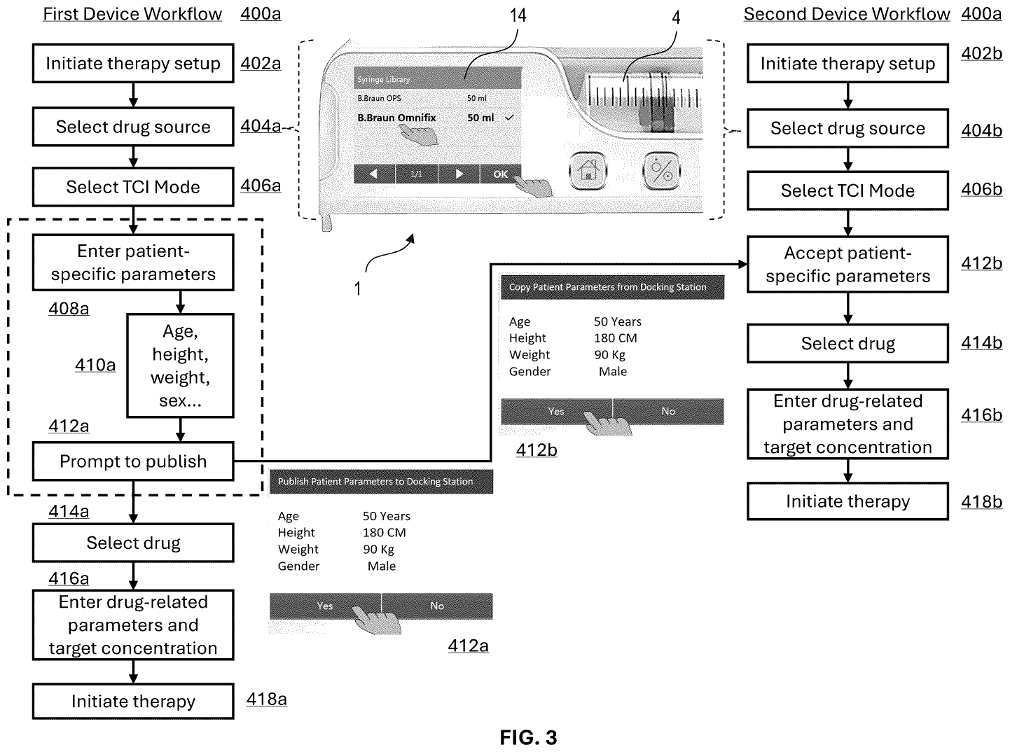

FIG. 3 depicts respective workflow processes 400a/b implemented by respective infusion devices to configure the infusion devices to perform a target controlled infusion (TCI), according to aspects of the subject technology. Each depicted workflow process involves a structured sequence of display screens and workflow steps enabling a clinician to enter patient data, select drugs and pharmacokinetic models, and initiate a model-driven infusion under defined safety and configuration constraints.

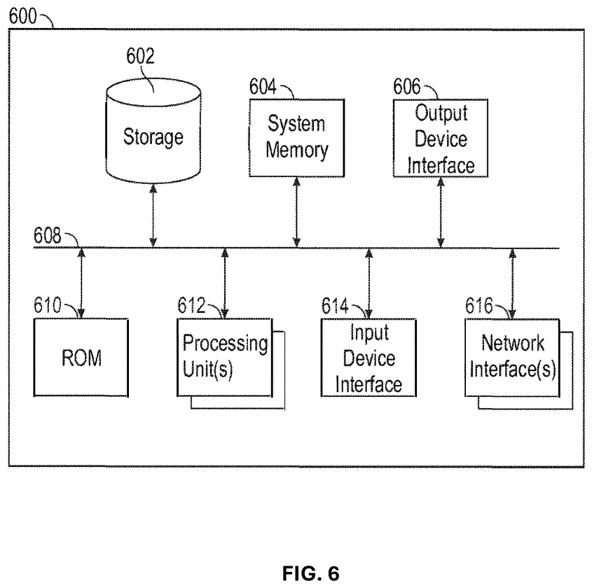

For explanatory purposes, the various blocks of example processes 400a/b are described herein with reference to FIGS. 1 through 3, and the components and/or processes described herein. One or more of the blocks of each process 400a/b may be implemented, for example, by an infusion device 1 (e.g., by way of computer control by the infusion device's electronic subsystems (see FIG. 6)).

In some implementations, one or more of the blocks of each process 400a/b may be implemented by a control unit 314 (e.g., of PCD 312 or docking station 300). In some implementations, one or more of the blocks of each process 400a/b may be implemented by a server for display by a display screen 14 of the infusion devices and/or a display of an associated connected computing device 332. For example, the infusion devices may communicate directly with the server or via control unit 314 and the server may perform the required processing operations and workflow steps and provide information for display on the respective display screens of the infusion devices and/or associated computing devices.

The disclosed TCI system may rely on pharmacokinetic (PK) and pharmacodynamic (PD) models that describe how the body absorbs, distributes, metabolizes, and eliminates the drug. These models may be encoded with control logic of an infusion pump and used to estimate drug concentration in real time. Based on estimates, the system may automatically increase or decrease the infusion rate to reach and maintain the clinician-specified target level.

The disclosed TCI system may further incorporate patient-specific information, such as age, sex, weight, height, and health status, to individualize the infusion parameters. The underlying pharmacokinetic model may use these patient-specific factors to estimate drug distribution and clearance rates, thereby improving dosing accuracy and safety. The result is a personalized and adaptive drug delivery system that reduces the need for constant manual adjustment by the clinician.

For explanatory purposes, the blocks of each example process 400a/b are described as occurring in serial, or linearly. However, the blocks of each example process 400a/b may occur in parallel. In addition, the blocks of each example process 400a/b need not be performed in the order shown and/or one or more of the blocks of each example process 400a/b need not be performed. Moreover, while the process is described below with regard to a first device workflow 400a of a first infusion device, the steps may be performed in the same or similar manner in the second device workflow 400b. As will be described further, the system provides a mechanism for quickly copying data for one or more workflow steps for retrieval by other subsequently performed workflows to quickly enter data and/or bypass workflow steps in the subsequently performed workflows.

Upon activation, each infusion device presents a startup screen that allows a clinician to initiate a therapy setup (402a/b). In this regard, the clinician may designate that the infusion being initiated involves a new patient. If the user indicates “New Patient,” the device transitions to a patient profile setup routine. Alternatively, the clinician may elect to retain a previously stored patient profile. The system may also incorporate a configurable “Clear Last Patient” function, set through a management software workstation (MSW), to ensure that prior patient information is deleted before a new TCI session begins.

The respective interface workflow may then prompt the clinician to select a drug source (404a/b). In the depicted example, a display screen 14 of the infusion device (e.g., a syringe pump 1) displays a syringe-selection screen presenting entries from a syringe library. The library may enumerate multiple drug source types, nominal volumes, and manufacturer or driver compatibility information as provisioned by the MSW. In the depicted example, selection of a syringe type from the library may then cause the controller to load syringe-specific calibration parameters, default VTBI values, and volumetric scaling factors for use in rate and volume computations. Having predetermined volumetric limits and calibration data associated with the chosen drug source type, the library step reduces the risk of mechanical-programming mismatch and constrains later volumetric inputs to values that are compatible with the selected drug source hardware.

After input of the drug source type is confirmed, the workflow may advance to one or more mode selection screens whereby, in the depicted example, the clinician selects the TCI mode (406a/b). The display screen of the infusion device may then display and prompt for selection of available TCI modes, for example plasma-targeted (Cp) or effect-site-targeted (Ce). In some implementations, the system may visually disable a mode that has been deactivated in the system configuration (e.g., a PCA mode may be disabled/greyed out).

Following mode selection, the respective graphic interface workflow seeks input of patient specific parameters. For the purposes of the depicted example, patient-specific parameters are entered in the first device workflow 400a and then rapidly copied to and retrieved by the second device workflow 400b. In this regard, the workflow steps receiving entry of the patient-specific parameters may then be bypassed/suppressed on the second infusion device, and any other devices (e.g., third and/or fourth infusion devices) that are operably connected to the first infusion device for providing a therapy to the same patient (e.g., via control module 314 of a PCD 312 or docking station 300). Moreover, while the following disclosure describes an example of copying of patient-specific parameters, the same process for other parameter sets may equally be implemented. For example, in some implementations, drug-related parameters and/or the desired target concentration may be copied and/or published and retrieved in the same manner.

Turning back to the first device workflow 400a depicted in FIG. 3, the system prompts the clinician to enter patient-specific physiological parameters (408a). The displayed input entry fields (410a) may include age, height, weight, and gender, and the like, for which the system may provide a different workflow interface screen for the prompting of each entry.

Accordingly, four entry screens may be displayed to obtain user input for age, height, weight, and gender. Additional screens may be provided for, for example, entry or confirmation of a patient's health condition (e.g., received from the patient's electronic medical record (EMR)).

Age may be, for example, entered in years or, for pediatric cases, in months, with permitted ranges typically between 1 and 88 years or 1 and 12 months. Height may be entered in centimeters, for example between 100 and 220 cm, and weight may be entered in kilograms, for example between 0.68 and 160 kg. The clinician provides user input for each displayed input field using chevron keys 16, 17 on display screen 14 of the infusion device 1. The infusion device may use these inputs to calculate derived values such as body mass index (BMI) or lean body mass, which may be subsequently used by a pharmacokinetic model to determine dosing parameters.

After the patient-specific parameters are entered in the first graphical interface workflow 400a of the first infusion device, the patient-specific parameters may be copied to the second infusion device to pre-complete a number of workflow steps of a second graphical interface workflow for programming and initiating a TCI (or other selected) therapy provided by the second infusion device (e.g., using a different drug). In this regard, the interface workflow may display (e.g., on display screen 14) a prompt to publish the patient-specific parameters (412a). The parameters may then be published for use by the second and other connected infusion devices involved in a therapy for a particular patient. In some implementations, the parameters are copied to a gateway server 330. In some implementations, the parameters are copied to a memory of the docking station 300.

In either case, when a second infusion device is connected to the docking station 314, the second infusion device may receive an indication that the parameters are available for download and the second infusion device may then display (e.g., on display screen 1) a prompt to copy the parameters to a memory of the second infusion device (412b), thereby pre-completing data entry for the same workflow steps in a second graphical interface workflow 300b of the second infusion device. In this regard, a number of workflow steps of the second interface workflow may be pre-completed and bypassed such that user input is not required for completion of the number of workflow steps of the second graphical interface workflow to program and initiate the therapy at the second infusion device. It is understood that, in such implementations, the number of workflow steps of the second graphical interface workflow would require user input had the set of parameters not been copied.

Returning to the respective graphical interface workflows 400a/b, after entry of patient information, the workflow may advance to drug selection (414a). The workflow may display a list of drugs available for TCI operation. The list of available drugs and their ordering may be defined in the MSW configuration, and multiple drugs (e.g., up to three) may be presented on a single workflow interface screen. In one example, the displayed drugs may include propofol, remifentanil, sufentanil, alfentanil, and dexmedetomidine, among others. Each drug may be associated with one or more pharmacokinetic models (e.g., the Eleveld model) and with one or more concentration formulations. If a predetermined MSW configuration defines only a single concentration for a given drug, a concentration selection screen may be omitted. Where multiple concentrations are available, the user may be prompted to select the desired concentration, such as 5, 10, or 20 mg/ml for propofol (not shown).

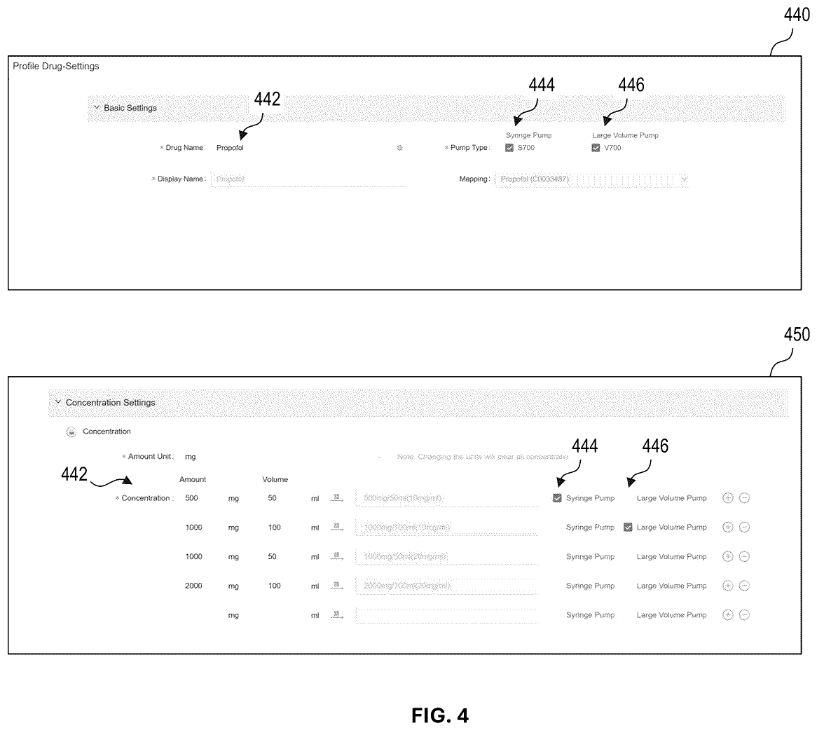

According to various implementations, each device workflow may suppress redundant screens/steps to streamline setup, for example, when only a single model or concentration exists. As will be described further, the number of options may be set in the disclosed MSW editor (see FIG. 4). If more than one drug model or concentration is configured, model names and concentration fields may be displayed to guide selection of the clinician. In some implementations, a therapy setup library may be received that includes default parameters for prepopulating a subset of the drug-related parameters in the respective workflow, and the default parameters may automatically populate one or more corresponding parameter fields within the respective workflow so that user input is not required to populate the parameter fields within the workflow. In this regard, at least one workflow step may be pre-completed so that user input is not required for completion of the workflow step. In some implementations, the therapy setup library may indicate workflow steps/screens which should be suppressed/bypassed.

Continuing with the device workflows of FIG. 3, following selection of the drug (and model), the interface workflow advances to a series of screens for entry of drug-related parameters and target concentration (416a/b). In this regard, the interface workflow may include a mode selection screen. The clinician may be able to choose between plasma-targeted (Cp) and effect-site-targeted (Ce) operation. If a target mode has been disabled in configuration, it may be displayed in grey and not selectable. A workflow screen may also allow entry of a desired target concentration (e.g., between 0.01 and 15.0μg/ml), as well as a maximum infusion rate (e.g., between 0.10 and 2000 ml/h). The maximum rate parameter may be defined in the MSW or editor software and, in some implementations, may be expressed as a volumetric rate or a dose-based rate, such as mg/kg/h.

In some implementations, additional selectable options in the workflow may include whether opioids are concurrently in use. Similarly, depending on the configuration, a volume-to-be-infused (VTBI) field may also be displayed, particularly when the pump is operating in volumetric mode. A default VTBI value may be established in the MSW.

Once all the configuration data are entered, the system may compute a predicted loading dose, induction rate, and maintenance rate using the selected pharmacokinetic model and the patient's entered parameters. A confirmation screen may then be displayed to summarize these computed values. For example, a summary screen may include BMI, induction dose and duration, induction rate and volume, and maintenance rate and duration. In one example therapy, the confirmation screen may display an induction dose of 1.79 mg/kg administered over 50 seconds, corresponding to a rate of 1200 ml/h and volume of 14.5 ml, followed by a maintenance phase of 64.3 ml/h for 150 seconds. The clinician reviews these values before confirming initiation of the infusion. A “Back” button may be provided to permit revision of any parameter prior to confirmation.

Upon confirmation, the system initiates infusion at a calculated rate designed to achieve and maintain the specified target concentration (418a/b). A display screen (e.g., the display screen 14 of the infusion device) may then provide continuous feedback on current plasma concentration (Cp), target or effect-site concentration (Ce or Ct), total dose delivered, and current flow rate. The display screen may also include indicators showing infusion status, mode (TCI, TIVA, or PCA), and alarm conditions.

During operation, if the clinician adjusts the target concentration, the control algorithm of the system may recalculate the infusion rate based on the active pharmacokinetic model. The system may be designed to provide a response time within approximately 0.5 seconds, avoiding latency that could otherwise lead to over-infusion or under-infusion.

All numerical entry fields may be subject to predefined limits established within the MSW configuration. These “guardrails” define acceptable ranges for target concentration, maximum rate, and cumulative dose, reducing the likelihood of programming errors. When a user attempts to enter a value outside an approved range, the pump may display an alert or prevent confirmation. The guardrail configuration can also enforce dose error reduction policies of a healthcare organization, ensuring that the infusion remains within predetermined safety limits.

Each graphical interface workflow may further include additional information screens summarizing session details such as total infused volume, operation duration, decay time and concentration, and patient demographic data. The display may show a time-evolving relationship between plasma and effect-site concentrations and an equilibrium indicator that identifies when target equilibration has been achieved.

In some implements, parameters may be automatically (e.g., without user intervention) and dynamically updated between devices during an ongoing therapy being jointly provided by the devices to the same patient. During an infusion, a parameter (e.g., a patient-specific parameter, drug-related parameter, or the desired target concentration) may be updated in a respective workflow of a respective infusion device of multiple infusion devices providing the therapy to the patient. For example, the parameter may be updated in the second graphical interface workflow 400b. Responsive to determining that the parameter was updated in the second graphical interface workflow, the system may notify the other infusion device(s) to display an alert of the update and/or prompt a clinician to update the parameter in the respective workflow(s) of the other device(s) to match the updated parameter. The clinician may then confirm the update at each respective device and the parameter is immediately copied and implemented in the ongoing TCI therapy on the respective device(s).

FIG. 4 depicts an example medication safety software (MSW) editor for generation of a therapy setup library for programming infusion devices for target controlled infusion, according to aspects of the subject technology. According to various implementations, the editor may comprise one or more, or a series of editing interface screens for setting default parameters and associated values in a given therapy to by implemented by the disclosed TCI system. While two example interface screens are depicted for entry of default drug-related parameters, other interface screens may also be implemented for setting other default parameters.

In some implementations, the editing interface screens may be generated by way of HTML or similar web-based languages transmission to various devices for access and data entry by a user clinician. In some implementations, the generation and transmission of the editing interface screens may be implemented by an information system server 330 for display on a remote computing device 332 (e.g., a mobile computing device associated with a clinician or respective infusion device docked in a docking station.) In some implementations, the editing interface screens may be generated by a mobile application on a computing device 332, with data being communicated by way of server 330 and a network 310, 340.

As an example, the MSW editor provides a therapy setup graphical user interface that allows a user to set default parameters for a respective target controlled infusion (TCI) therapy. The user sets the parameters, and what types of infusion devices the parameters apply to, and the editor software generates a therapy setup library which may be downloaded to respective infusion devices for implementation.

In the depicted example, a first interface screen 440 includes a drug selection control 442 to allow a user to select a drug for which default parameters will be set. One or more device selection controls 444, 446 identify what types of medical devices the parameters will be applied to. In the depicted example, both syringe pump and large volume pump are selected, meaning that the default values entered (in subsequent screens) for the selected drug (propofol) will be automatically set in both syringe pumps and large volume pumps that utilize the resulting therapy setup library. While drugs are setup in the editor may be reflected in the drug selection screens (414a/b) discussed with respect to FIG. 3.

The second interface screen 450 includes a number of inputs 452 for setting concentration options, and respective selection controls 444, 446 to identify what types of medical devices the parameters of each concentration setting will be applied to. In this regard, a user may define which drug concentration options are available for selection in a corresponding graphical interface workflow, such as within the series of screens for entry of drug-related parameters and target concentration (416a/b) shown in FIG. 3. The user may define one or more concentration options for each type of infusion device. In the depicted example, only one option (500 mg/50 ml) is populated for syringe pump and one option (1000 mg/100 ml) is populated for large volume pump. Accordingly, when utilized in the respective pumps, and the workflow (416a/b) is displayed, only one option will be available for selection by the user.

Accordingly, selecting syringe pump in the second interface screen 450 causes the default parameters to be applied to syringe pumps to populate corresponding parameter fields within the respective graphical interface workflow of each syringe pump (FIG. 3) so that user input is not required to populate parameter fields within the respective workflow. Similarly, selecting large volume pump in the second interface screen 450 causes the corresponding default parameters to be applied to large volume pumps to populate corresponding parameter fields within the respective graphical interface workflow of each large volume pump so that user input is not required to populate parameter fields within the workflow.

Moreover, the selections made in the MSW editor may determine what options are available in each respective workflow, which may result in a reduction of, or bypassing of, workflow steps. For example, if an infusion device type is associated with only one option (e.g., one drug type) for a particular screen (e.g., selecting a drug) then the workflow implementing the therapy setup library may omit steps pertaining to the selection of available options (e.g., the drug type).

A therapy setup library may include first default parameters for prepopulating a first subset of the drug-related parameters in a respective workflow of a first type of infusion device and second default parameters for prepopulating a second subset of the drug-related parameters in a respective workflow of a second type of the infusion device.

In a particular therapy, it may be determined that a first infusion device is of the first type of infusion device, and a second infusion device is of a second type of infusion device. Responsive to receiving the therapy setup library, the first default parameters may be caused to be stored in the first infusion device to populate one or more corresponding parameter fields within a first graphical interface workflow of the first infusion device so that user input is not required to populate the one or more corresponding parameter fields within the first graphical interface workflow. Likewise, the second default parameters may be caused to be stored in the second infusion device to populate one or more corresponding parameter fields within a second graphical interface workflow of the second infusion device so that user input is not required to populate the one or more corresponding parameter fields within the second graphical interface workflow.

As described previously, prepopulating of parameter fields may result in a selection screen or input screen to no longer be needed. In this regard, workflow steps that are prepopulated with data or having a sole selection resulting from selections made at the MSW editor may not longer be needed and may be bypassed, leading to a more efficient setup of the infusion device. In some implementations, the MSW editor may allow a user to set a flag to bypass the corresponding workflow step, or the flag may be automatically set when the step is prepopulated, and meta data may be sent with the library indicating whether the workflow step is to be bypassed in the respective workflow.

Accordingly, in some implementations, populating the one or more corresponding parameter fields within the first graphical interface workflow with the first default parameters may cause at least one workflow step of the first graphical interface workflow to be pre-completed and/or bypassed so that user input is not required for completion of the at least one workflow step of the first graphical interface workflow. Likewise, populating the one or more corresponding parameter fields within the second graphical interface workflow with the first default parameters may cause at least one workflow step of the second graphical interface workflow to be pre-completed and/or bypassed so that user input is not required for completion of the at least one workflow step of the second graphical interface workflow.

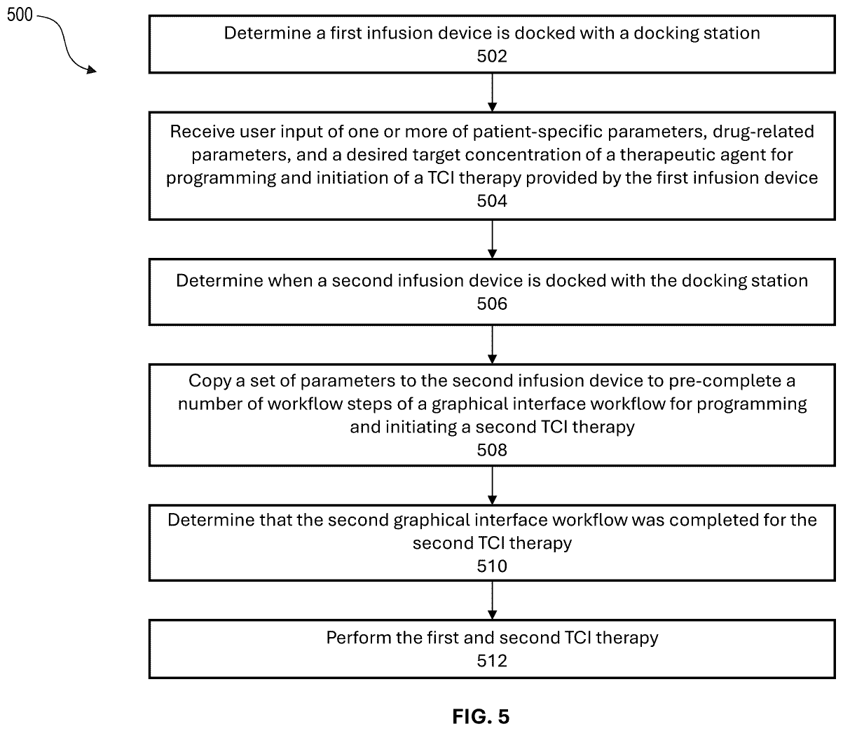

FIG. 5 depicts an example process 500 for programming infusion devices for target controlled infusion, according to aspects of the subject technology. For explanatory purposes, the various blocks of example processes 500 are described herein with reference to FIGS. 1 through 4, and the components and/or processes described herein.

In some implementations, one or more of the blocks of process 500 may be implemented by a control unit 314 (e.g., of PCD 312 or docking station 300). In some implementations, one or more of the blocks of process 500 may be implemented by a server for display by a display screen 14 of the infusion devices and/or a display of an associated connected computing device 332. For example, the infusion devices may communicate directly with the server or via control unit 314 and the server may perform the required processing operations for the disclosed step(s). In some implementations, one or more of the blocks of process 500 may be implemented, for example, by an infusion device 1. For example, one or more of the blocks may be implemented based on computer control by the infusion device's electronic subsystems (see, e.g., FIG. 6).

For explanatory purposes, the blocks of process 500 are described as occurring in serial, or linearly. However, the blocks of process 500 may occur in parallel. In addition, the blocks of process 500 need not be performed in the order shown and/or one or more of the blocks of process 500 need not be performed.

As described previously, multiple infusion devices (e.g., syringe pumps 1) may be operably connected to each other by way of being docked to a docking station 300 (and/or control module 314) to provide one or more therapies to the same patient. The devices may both be docked prior to a therapy being initiated for the patient or one or more devices may be docked to participate in an ongoing therapy. For the purposes of disclosure, the infusion devices are programmed for target controlled infusions. A first infusion device is programmed to deliver a first drug to achieve a first target drug concentration in the patient and a second infusion device is programmed to deliver a second drug to achieve a second target concentration in the patient.

In an example in which the therapy delivered to induce and maintain anesthesia, a first device may deliver a hypnotic agent such as propofol, and the other may deliver an opioid analgesic such as remifentanil. Each infusion device may operate under a separate target controlled infusion (TCI) algorithm based on a pharmacokinetic model specific to the drug, and calculate and continuously adjust infusion rates to achieve and maintain predetermined (e.g., clinician set) target drug concentrations in the patient (e.g., in the brain).

In some implementations, the system may coordinate the infusion devices to adjust one drug's target concentration (e.g., propofol) in response to changes in the other drug (e.g., remifentanil) to maintain a balanced depth of anesthesia and prevent excessive sedation or analgesia. In this regard, a clinical decision algorithm may receive sensor measurements and/or data from a sensor coupled to the patient, determine a state of the patient based on those measurements and/or data, and adjust the target concentration of the drug delivered by each connected infusion device to keep the patient in a particular state.

For example, during anesthesia, the system may receive electroencephalography (EEG) data (e.g., bispectral index values) indicating a depth of the anesthesia, and electrocardiography (ECG) data indicative of an autonomic response (e.g., heart rate and/or blood pressure). If bispectral values rise above a predetermined threshold range for hypnosis while heart rate and blood pressure increase, the algorithm may automatically increase the propofol target concentration. If the BIS is acceptable but heart rate or blood pressure (e.g., derived from ECG signals and/or blood pressure monitoring) rise above a target range then the algorithm may automatically increase the remifentanil target concentration. Similarly, propofol may be reduced if the BIS value falls below the target range and heart rate and blood pressure drop (indicating excessive anesthesia), and remifentanil may be reduced if heart rate or blood pressure fall below target while BIS remains within range or low (indicating excessive analgesia).

With reference to FIG. 5, the process 500 starts with the system determining that a first infusion device is docked with a docking station configured to receive a plurality of infusion devices (502). In this regard, a control module 314 may monitor a connection bus within a docking station 300 for signals provided by respective infusion devices to the docking station when they become docked to the docking station. It is understood that docking Additionally or in the alternative, respective infusion devices may become operably connected to the docking station and/or control module 314 by way of a pairing mechanism (e.g., BLUETOOTH), WiFi, or cabling (e.g., Ethernet, USB, or serial cable).

The system receives user input of one or more of patient-specific parameters, drug-related parameters, and a desired target concentration of a therapeutic agent for programming and initiation of a first target controlled infusion (TCI) therapy provided by the first infusion device (502). As described with respect to FIG. 4, these parameters may be received via a first graphical interface workflow displayed on the first infusion device. In some implementations, the parameters may be received by a computing device (e.g., a mobile device) associated with the first infusion device (e.g., by way of being paired with the infusion device or connected to the infusion device via the docking station).

The system determines when a second infusion device becomes docked with the docking station (506). This determination may be performed in any one of the manners disclosed previously with regard to the first infusion device. It is understood that docking of the first and/or second infusion device can be detected at any time. The system detects the docking and when more than one infusion device is docked the system coordinates communications between the connected devices.

After the patient-specific parameters, the drug-related parameters, or the desired target concentration are received (and after determining that the second infusion device is docked), a set of parameters is copied to the second infusion device to pre-complete a number of workflow steps of a second graphical interface workflow for programming and initiating a second TCI therapy provided by the second infusion device (508). As described previously, the set of parameters may include the patient-specific parameters, or a set of parameters from the drug-related parameters or the target concentration. For the purposes of this example, a set of patient-specific parameters is copied.

To initiate copying of the parameters, the workflow of the first infusion device may prompt the user to publish the parameters. Publishing the set of parameters makes the parameters available to other devices that are in communication with the first infusion device (e.g., by way of being docked to the same docking station and/or control unit or by way of being in communication with the first infusion device). Accordingly, the first infusion device may display, on its display screen 14, a prompt for allowing the user to confirm whether the parameters should be published. As described with regard to step 412a, the prompt may be provided in a graphic interface that provides the user the opportunity to confirm or reject publication/copying. The set of parameters is copied responsive to receiving a confirmation of the prompt.

In some implementations, the copying the set of parameters to the second infusion device includes the first infusion device copying the set of parameters to a gateway server (e.g., server 330) and the second infusion device retrieving the set of parameters from the gateway server. In some implementations, copying the set of parameters to the second infusion device includes the first infusion device copying the set of parameters to a memory of the docking station (e.g., disk 356 and/or a memory of control unit 314), and the docking station providing an indication that the set of parameters are available to the second infusion device. The second infusion device may then receive the indication and retrieve the set of parameters from the memory of the docking station.

According to this example, the prompt is displayed after receiving the patient-specific parameters, including one or more of an age, weight, sex, height, and a health condition of the patient being treated. In this regard, a clinician programming another docked/connected infusion device may immediately accept the published parameters for copying to the device to skip/bypass steps in a corresponding workflow on the device that would otherwise be required to input the same parameters for the same patient.

In some implementations, the data will be prepopulated, and the user merely skips through the screens, confirming the data. In some implementations, the workflow steps/screens that are prepopulated are automatically bypassed/suppressed. In some implementations, the received/copied data may include meta data indicating which workflow steps in the workflow (e.g., when the workflows are the same) may be bypassed/suppressed. Accordingly, the clinician may only have to navigate the prompting screen instead of a multitude of screens required to input and/or confirm the data.

Accordingly, after the set of parameters is copied to the second infusion device and the second graphical interface workflow is displayed on a display screen of the second infusion device, a number of workflow steps of the second graphical interface workflow may be pre-completed and bypassed such that user input is not required for completion of the number of workflow steps of the second graphical interface workflow to program and initiate the second TCI therapy. In this example pertaining to patient-specific parameters, the number of workflow steps that are pre-completed and bypassed on the second graphical interface workflow include workflow steps for receiving user input of the patient-specific parameters.

Continuing with the example process 500, the system determines that the second graphical interface workflow was completed for the second TCI therapy (510), and the first and/or second TCI therapies are performed (512). It is understood that the therapies of the different devices need not be linked, and each device may begin its infusion independently. In some implementations, a central control algorithm manages the infusions and may wait until all infusion devices are ready before beginning the therapies.

In some implementations, during an ongoing therapy (e.g., while a first drug is being provided by the first infusion device and a second drug is being provided by a second infusion device), the system may determine that at least one parameter (e.g., a patient-specific parameter of the patient-specific parameters) was updated in a respective graphical interface workflow of a respective infusion device. For the purpose of this example, the at least one parameter is updated in the second graphical interface workflow of the second infusion device. Each infusion device may be configured to report updates to its parameter(s) via a bus communication with the docking station and/or control unit 314. In this manner, the update may be published to all connected/docked infusion devices.

Responsive to determining that the at least one parameter (e.g., patient-specific parameter) was updated in the second graphical interface workflow of the second infusion device, the first infusion device may display a prompt for updating the at least one patient-specific parameter in the first graphical interface workflow of the first infusion device. In this manner, the same parameter(s) in the first infusion device may be updated to match the parameter(s) updated in the second graphical interface workflow of the second infusion device. The prompt displayed to confirm the updating may be displayed in the same or similar manner as the prompt 412b, previously described.

Accordingly, a confirmation of the prompt is received in the first infusion device workflow and, responsive to the confirmation, the at least one parameter is automatically updated in the first graphical interface workflow of the first infusion device to match the at least one patient-specific parameter updated in the second graphical interface workflow of the second infusion device. In some implementations, upon receiving the confirmation, the parameter(s) may be updated without entering into the workflow process. In some implementations, updating the parameter(s) may initiate the workflow process, for example, at a workflow step corresponding to entry of the parameter(s).

The system also provides a mechanism for ensuring user compliance with loading of medication. It has been suggested that when multiple pump systems are in operation there is a significant chance of a user erroneously loading a medication in the wrong device. Accordingly, the system (e.g., each infusion device) is configured to display a human perceptible indicator indicative of the medication for which it is programmed, particularly when the device is in a loading mode (e.g., when in standby mode).

Accordingly, each infusion device workflow may assign a predetermined graphic to each drug type. When a drug type is programmed into the pump (e.g., propofol), the predetermined graphic for the drug is set in the workflow. When the infusion device is placed in a loading mode, the system receives an indication for loading the medication source (e.g., a syringe) and, responsive to the indication, changes the workflow interface screen to display the predetermined graphic for the proper medication, at least until the medication source is loaded. In some implementations, the predetermined graphic includes changing the color of the entire screen or at least a substantial portion of the screen to a particular color associated with the drug type. The display thus provides a fallback mechanism for and/or alleviates reliance on other more costly mechanisms such as RFID enabled medication sources and RFID readers embedded within the pump.

Many of the above-described devices, systems and processes (e.g., processes 400a/b and 500), may also be controlled by software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

FIG. 6 is a conceptual diagram illustrating an example electronic system 600 for programming infusion devices for target controlled infusion, according to aspects of the subject technology. Electronic system 600 may be a computing device for execution of software associated with one or more components and processes provided by FIGS. 1-5, including but not limited to infusion device 1, computing device 312, control unit 314, functional modules 316-322, docking station 300, or server 330, or computing hardware within these devices and/or systems. Electronic system 600 may include a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 600 may include various types of computer readable media and interfaces for various other types of computer readable media. In the depicted example, electronic system 600 includes a bus 608, processing unit(s) 612, a system memory 604, a read-only memory (ROM) 610, a permanent storage device 602, an input device interface 614, an output device interface 606, and one or more network interfaces 616. In some implementations, electronic system 600 may include or be integrated with other computing devices or circuitry for operation of the various components and processes previously described.

Bus 608 collectively represents all system, peripheral, and chipset buses that communicatively connect the numerous internal devices of electronic system 600. For instance, bus 608 communicatively connects processing unit(s) 612 with ROM 610, system memory 604, and permanent storage device 602.

From these various memory units, processing unit(s) 612 retrieves instructions to execute and data to process in order to execute the processes of the subject disclosure. The processing unit(s) can be a single processor or a multi-core processor in different implementations.

ROM 610 stores static data and instructions that are needed by processing unit(s) 612 and other modules of the electronic system. Permanent storage device 602, on the other hand, is a read-and-write memory device. This device is a non-volatile memory unit that stores instructions and data even when electronic system 600 is off. Some implementations of the subject disclosure use a mass-storage device (such as a magnetic or optical disk and its corresponding disk drive) as permanent storage device 602.

Other implementations use a removable storage device (such as a floppy disk, flash drive, and its corresponding disk drive) as permanent storage device 602. Like permanent storage device 602, system memory 604 is a read-and-write memory device. However, unlike storage device 602, system memory 604 is a volatile read-and-write memory, such a random access memory. System memory 604 stores some of the instructions and data that the processor needs at runtime. In some implementations, the processes of the subject disclosure are stored in system memory 604, permanent storage device 602, and/or ROM 610. From these various memory units, processing unit(s) 612 retrieves instructions to execute and data to process in order to execute the processes of some implementations.

Bus 608 also connects to input and output device interfaces 614 and 606. Input device interface 614 enables the user to communicate information and select commands to the electronic system. Input devices used with input device interface 614 include, e.g., alphanumeric keyboards and pointing devices (also called “cursor control devices”). Output device interfaces 606 enables, e.g., the display of images generated by the electronic system 600. Output devices used with output device interface 606 include, e.g., printers and display devices, such as cathode ray tubes (CRT) or liquid crystal displays (LCD). Some implementations include devices such as a touchscreen that functions as both input and output devices.

Also, as shown in FIG. 6, bus 608 also couples electronic system 600 to a network (not shown) through network interfaces 616. Network interfaces 616 may include, e.g., a wireless access point (e.g., Bluetooth or WiFi) or radio circuitry for connecting to a wireless access point. Network interfaces 616 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 600 can be used in conjunction with the subject disclosure.

These functions described above can be implemented in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification.
Clause 1. A machine implemented method for programming infusion devices for target controlled infusion, comprising: determining a first infusion device is docked with a docking station configured to receive a plurality of infusion devices; receiving, via a first graphical interface workflow displayed on the first infusion device, user input of one or more of patient-specific parameters, drug-related parameters, and a desired target concentration of a therapeutic agent for programming and initiation of a first target controlled infusion (TCI) therapy provided by the first infusion device; determining when a second infusion device becomes docked with the docking station; and after receiving the patient-specific parameters, the drug-related parameters, or the desired target concentration, and responsive to determining that the first and second infusion devices are simultaneously docked with the docking station, copying a set of parameters to the second infusion device to pre-complete a number of workflow steps of a second graphical interface workflow for programming and initiating a second TCI therapy provided by the second infusion device; wherein, after copying the set of parameters to the second infusion device and the second graphical interface workflow is displayed on a display screen of the second infusion device, the number of workflow steps of the second graphical interface workflow are pre-completed and bypassed such that user input is not required for completion of the number of workflow steps of the second graphical interface workflow to program and initiate the second TCI therapy, wherein the number of workflow steps of the second graphical interface workflow would require user input had the set of parameters not been copied.
Clause 2. The machine implemented method of Clause 1, further comprising: causing the second infusion device to display the second graphical interface workflow on the display screen of the second infusion device; receiving an indication that the second graphical interface workflow was completed on the second infusion device for programming and initiation of a second TCI therapy provided by the second infusion device; and causing the second infusion device to initiate the second TCI therapy based on the set of parameters copied to the second infusion device.
Clause 3. The machine implemented method of Clause 1, wherein copying the set of parameters to the second infusion device comprises: the first infusion device copying the set of parameters to a gateway server; and the second infusion device retrieving the set of parameters from the gateway server.
Clause 4. The machine implemented method of Clause 1, wherein copying the set of parameters to the second infusion device comprises: the first infusion device copying the set of parameters to a memory of the docking station; and the docking station providing an indication that the set of parameters are available to the second infusion device; and the second infusion device receiving the indication and retrieving the set of parameters from the first infusion device from the memory of the docking station.
Clause 5. The machine implemented method of Clause 1, further comprising, after the second infusion device becomes docked with the docking station and before the set of parameters is copied: causing the first infusion device to display, on a display screen of the first infusion device, a prompt to confirm the copying of the set of parameters, wherein the set of parameters is copied responsive to receiving a confirmation of the prompt.
Clause 6. The machine implemented method of Clause 5, further comprising: causing the prompt to be displayed after receiving the patient-specific parameters, wherein the patient-specific parameters include one or more of an age, weight, sex, height, and a health condition, and wherein the number of workflow steps that are pre-completed and bypassed on the second graphical interface workflow comprise workflow steps for receiving user input of the patient-specific parameters.
Clause 7. The machine implemented method of Clause 1, further comprising: receiving a drug type via the first graphical interface workflow; assigning a predetermined graphic to the received drug type; receiving an indication for loading a medication source into the first infusion device; and responsive to the indication, changing the first graphical interface workflow to display the predetermined graphic at least until the medication source is loaded.
Clause 8. The machine implemented method of Clause 7, wherein receiving the indication for loading the medication source comprises receiving a request to place the first infusion device in a standby mode; wherein assigning the predetermined graphic to the received drug type comprises assigning a predetermined color to the received drug type; and wherein changing the first graphical interface workflow to display the predetermined graphic comprises changing a current color of at least a portion of the display screen of the first infusion device from a first color to the assigned screen color.
Clause 9. The machine implemented method of Clause 1, further comprising: determining, while the first TCI therapy is being provided by the first infusion device, that at least one patient-specific parameter of the patient-specific parameters was updated in the second graphical interface workflow of the second infusion device; and responsive to determining that the at least one patient-specific parameter of the patient-specific parameters was updated in the second graphical interface workflow of the second infusion device, causing the first infusion device to display a prompt for updating the at least one patient-specific parameter in the first graphical interface workflow of the first infusion device to match the at least one patient-specific parameter updated in the second graphical interface workflow of the second infusion device; receiving a confirmation of the prompt; and responsive to the confirmation, automatically updating the at least one patient-specific parameter in the first graphical interface workflow of the first infusion device to match the at least one patient-specific parameter updated in the second graphical interface workflow of the second infusion device.
Clause 10. The machine implemented method of Clause 1, further comprising: receiving, from a server remote from the docking station, a therapy setup library comprising first default parameters for prepopulating a first subset of the drug-related parameters in a respective workflow of a first type of infusion device and second default parameters for prepopulating a second subset of the drug-related parameters in a respective workflow of a second type of the infusion device; responsive to receiving the therapy setup library: determining that the first infusion device is of the first type of infusion device and the second infusion device is of the second type of infusion device; causing the first default parameters to be stored in the first infusion device to populate one or more corresponding parameter fields within the first graphical interface workflow so that user input is not required to populate the one or more corresponding parameter fields within the first graphical interface workflow; and causing the second default parameters to be stored in the second infusion device to populate one or more corresponding parameter fields within the second graphical interface workflow so that user input is not required to populate the one or more corresponding parameter fields within the second graphical interface workflow.
Clause 11. The machine implemented method of Clause 10, wherein populating the one or more corresponding parameter fields within the first graphical interface workflow with the first default parameters causes at least one workflow step of the first graphical interface workflow to be pre-completed so that user input is not required for completion of the at least one workflow step of the first graphical interface workflow, and wherein populating the one or more corresponding parameter fields within the second graphical interface workflow with the first default parameters causes at least one workflow step of the second graphical interface workflow to be pre-completed so that user input is not required for completion of the at least one workflow step of the second graphical interface workflow.
Clause 12. The machine implemented method of Clause 1, wherein the first infusion device and the second infusion device comprise a syringe pump or a large volume pump.
Clause 13. A system for programming infusion devices for target controlled infusion, comprising: a docking station; and one or more processors configured to: determine a first infusion device docked with a docking station configured to receive a plurality of infusion devices; receive, via a first graphical interface workflow displayed on the first infusion device, user input of one or more of patient-specific parameters, drug-related parameters, and a desired target concentration of a therapeutic agent for programming and initiation of a first target controlled infusion (TCI) therapy provided by the first infusion device; determine when a second infusion device becomes docked with the docking station; and after receiving the patient-specific parameters, the drug-related parameters, or the desired target concentration, and responsive to determining that the first and second infusion devices are simultaneously docked with the docking station, copy a set of parameters to the second infusion device to pre-complete a number of workflow steps of a second graphical interface workflow for programming and initiating a second TCI therapy provided by the second infusion device; wherein, after copying the set of parameters to the second infusion device and the second graphical interface workflow is displayed on a display screen of the second infusion device, the number of workflow steps of the second graphical interface workflow are pre-completed and bypassed such that user input is not required for completion of the number of workflow steps of the second graphical interface workflow to program and initiate the second TCI therapy, wherein the number of workflow steps of the second graphical interface workflow would require user input had the set of parameters not been copied.
Clause 14. The system of Clause 13, wherein the one or more processors are further configured to: receiving an indication that the second graphical interface workflow was completed on the second infusion device for programming and initiation of a second TCI therapy provided by the second infusion device; and causing the second infusion device to initiate the second TCI therapy based on the set of parameters copied to the second infusion device.
Clause 15. The system of Clause 13, wherein the one or more processors are further configured to, after the second infusion device becomes docked with the docking station and before the set of parameters is copied: cause the first infusion device to display, on a display screen of the first infusion device, a prompt to confirm the copying of the set of parameters, wherein the set of parameters is copied responsive to receiving a confirmation of the prompt.
Clause 16. The system of Clause 15, wherein the one or more processors are further configured to: cause the prompt to be displayed after receiving the patient-specific parameters, wherein the patient-specific parameters include one or more of an age, weight, sex, height, and a health condition, and wherein the number of workflow steps that are pre-completed and bypassed on the second graphical interface workflow comprise workflow steps for receiving user input of the patient-specific parameters.
Clause 17. The system of Clause 13, wherein the one or more processors are further configured to: receive a request to place the first infusion device in a standby mode; assign a predetermined color to the received drug type; and change a current color of at least a portion of the display screen of the first infusion device from a first color to the assigned screen color.
Clause 18. The system of Clause 13, wherein the one or more processors are further configured to: determine, while the first TCI therapy is being provided by the first infusion device, that at least one patient-specific parameter of the patient-specific parameters was updated in the second graphical interface workflow of the second infusion device; and responsive to determining that the at least one patient-specific parameter of the patient-specific parameters was updated in the second graphical interface workflow of the second infusion device, cause the first infusion device to display a prompt for updating the at least one patient-specific parameter in the first graphical interface workflow of the first infusion device to match the at least one patient-specific parameter updated in the second graphical interface workflow of the second infusion device; receive a confirmation of the prompt; and responsive to the confirmation, automatically update the at least one patient-specific parameter in the first graphical interface workflow of the first infusion device to match the at least one patient-specific parameter updated in the second graphical interface workflow of the second infusion device.
Clause 19. The system of Clause 13, wherein the one or more processors are further configured to: receive, from a server remote from the docking station, a therapy setup library comprising first default parameters for prepopulating a first subset of the drug-related parameters in a respective workflow of a first type of infusion device and second default parameters for prepopulating a second subset of the drug-related parameters in a respective workflow of a second type of the infusion device; responsive to receiving the therapy setup library: determine that the first infusion device is of the first type of infusion device and the second infusion device is of the second type of infusion device; cause the first default parameters to be stored in the first infusion device to populate one or more corresponding parameter fields within the first graphical interface workflow so that user input is not required to populate the one or more corresponding parameter fields within the first graphical interface workflow; and cause the second default parameters to be stored in the second infusion device to populate one or more corresponding parameter fields within the second graphical interface workflow so that user input is not required to populate the one or more corresponding parameter fields within the second graphical interface workflow.
Clause 20. A non-transitory machine readable medium storing instructions thereon that, when executed by one or more processors, cause the one or more processors to perform operations comprising: determining a first infusion device docked with a docking station configured to receive a plurality of infusion devices; receiving, via a first graphical interface workflow displayed on the first infusion device, user input of one or more of patient-specific parameters, drug-related parameters, and a desired target concentration of a therapeutic agent for programming and initiation of a first target controlled infusion (TCI) therapy provided by the first infusion device; determining when a second infusion device becomes docked with the docking station; and after receiving the patient-specific parameters, the drug-related parameters, or the desired target concentration, and responsive to determining that the first and second infusion devices are simultaneously docked with the docking station, copying a set of parameters to the second infusion device to pre-complete a number of workflow steps of a second graphical interface workflow for programming and initiating a second TCI therapy provided by the second infusion device; wherein, after copying the set of parameters to the second infusion device and the second graphical interface workflow is displayed on a display screen of the second infusion device, the number of workflow steps of the second graphical interface workflow are pre-completed and bypassed such that user input is not required for completion of the number of workflow steps of the second graphical interface workflow to program and initiate the second TCI therapy, wherein the number of workflow steps of the second graphical interface workflow would require user input had the set of parameters not been copied.

### Further Considerations

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable a person of ordinary skill in the art to practice the various aspects described herein. While the foregoing has described what are considered to be the best mode and/or other examples, it is understood that various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more. ” Unless specifically stated otherwise, the terms “a set” and “some” refer to one or more. Pronouns in the masculine (e.g., his) include the feminine and neuter gender (e.g., her and its) and vice versa. Headings and subheadings, if any, are used for convenience only and do not limit the invention.

The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration. ” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such an embodiment may refer to one or more embodiments and vice versa.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, C, C++, web services, or rich site summary (RSS). In some embodiments, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

The word “exemplary” is used herein to mean “serving as an example or illustration.” Any aspect or design described herein as “exemplary” is not necessarily to be construed as preferred or advantageous over other aspects or designs.

As used herein, a “key” that can be pressed may be implemented as an interactive element on a device that specifically causes the identified response or a general purpose interactive element that, depending on operational state of the device, causes the identified response. In some implementations, the interactive element may be an electromechanical element (e.g., button or switch) to generate a signal or message. In some implementations, the interactive element may be a virtual element presented on a graphical user interface (e.g., button, slider, etc.) that, when interacted with, generates a signal or message.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

All structural and functional equivalents to the elements of the various aspects described throughout this disclosure that are known or later come to be known to those of ordinary skill in the art are expressly incorporated herein by reference and are intended to be encompassed by any claim. Furthermore, to the extent that the term “include,” “have,” or the like is used in the description, such term is intended to be inclusive in a manner similar to the term “comprise” as “comprise” is interpreted when employed as a transitional word.

What is claimed is:

1. A machine implemented method for programming infusion devices for target controlled infusion, comprising:
determining a first infusion device is docked with a docking station configured to receive a plurality of infusion devices;
receiving, via a first graphical interface workflow displayed on the first infusion device, user input of one or more of patient-specific parameters, drug-related parameters, and a desired target concentration of a therapeutic agent for programming and initiation of a first target controlled infusion (TCI) therapy provided by the first infusion device;
determining when a second infusion device becomes docked with the docking station; and
after receiving the patient-specific parameters, the drug-related parameters, or the desired target concentration, and responsive to determining that the first and second infusion devices are simultaneously docked with the docking station, copying a set of parameters to the second infusion device to pre-complete a number of workflow steps of a second graphical interface workflow for programming and initiating a second TCI therapy provided by the second infusion device;
wherein, after copying the set of parameters to the second infusion device and the second graphical interface workflow is displayed on a display screen of the second infusion device, the number of workflow steps of the second graphical interface workflow are pre-completed and bypassed such that user input is not required for completion of the number of workflow steps of the second graphical interface workflow to program and initiate the second TCI therapy, wherein the number of workflow steps of the second graphical interface workflow would require user input had the set of parameters not been copied.

2. The machine implemented method of claim 1, further comprising:
causing the second infusion device to display the second graphical interface workflow on the display screen of the second infusion device;
receiving an indication that the second graphical interface workflow was completed on the second infusion device for programming and initiation of a second TCI therapy provided by the second infusion device; and
causing the second infusion device to initiate the second TCI therapy based on the set of parameters copied to the second infusion device.

3. The machine implemented method of claim 1, wherein copying the set of parameters to the second infusion device comprises:
the first infusion device copying the set of parameters to a gateway server; and
the second infusion device retrieving the set of parameters from the gateway server.

4. The machine implemented method of claim 1, wherein copying the set of parameters to the second infusion device comprises:
the first infusion device copying the set of parameters to a memory of the docking station; and
the docking station providing an indication that the set of parameters are available to the second infusion device; and
the second infusion device receiving the indication and retrieving the set of parameters from the first infusion device from the memory of the docking station.

5. The machine implemented method of claim 1, further comprising, after the second infusion device becomes docked with the docking station and before the set of parameters is copied:
causing the first infusion device to display, on a display screen of the first infusion device, a prompt to confirm the copying of the set of parameters,
wherein the set of parameters is copied responsive to receiving a confirmation of the prompt.

6. The machine implemented method of claim 5, further comprising:
causing the prompt to be displayed after receiving the patient-specific parameters,
wherein the patient-specific parameters include one or more of an age, weight, sex, height, and a health condition, and
wherein the number of workflow steps that are pre-completed and bypassed on the second graphical interface workflow comprise workflow steps for receiving user input of the patient-specific parameters.

7. The machine implemented method of claim 1, further comprising:
receiving a drug type via the first graphical interface workflow;
assigning a predetermined graphic to the received drug type;
receiving an indication for loading a medication source into the first infusion device; and
responsive to the indication, changing the first graphical interface workflow to display the predetermined graphic at least until the medication source is loaded.

8. The machine implemented method of claim 7,
wherein receiving the indication for loading the medication source comprises receiving a request to place the first infusion device in a standby mode;
wherein assigning the predetermined graphic to the received drug type comprises assigning a predetermined color to the received drug type; and
wherein changing the first graphical interface workflow to display the predetermined graphic comprises changing a current color of at least a portion of the display screen of the first infusion device from a first color to the assigned screen color.

9. The machine implemented method of claim 1, further comprising:
determining, while the first TCI therapy is being provided by the first infusion device, that at least one patient-specific parameter of the patient-specific parameters was updated in the second graphical interface workflow of the second infusion device; and
responsive to determining that the at least one patient-specific parameter of the patient-specific parameters was updated in the second graphical interface workflow of the second infusion device, causing the first infusion device to display a prompt for updating the at least one patient-specific parameter in the first graphical interface workflow of the first infusion device to match the at least one patient-specific parameter updated in the second graphical interface workflow of the second infusion device;
receiving a confirmation of the prompt; and
responsive to the confirmation, automatically updating the at least one patient-specific parameter in the first graphical interface workflow of the first infusion device to match the at least one patient-specific parameter updated in the second graphical interface workflow of the second infusion device.

10. The machine implemented method of claim 1, further comprising:
receiving, from a server remote from the docking station, a therapy setup library comprising first default parameters for prepopulating a first subset of the drug-related parameters in a respective workflow of a first type of infusion device and second default parameters for prepopulating a second subset of the drug-related parameters in a respective workflow of a second type of the infusion device;
responsive to receiving the therapy setup library:
determining that the first infusion device is of the first type of infusion device and the second infusion device is of the second type of infusion device;
causing the first default parameters to be stored in the first infusion device to populate one or more corresponding parameter fields within the first graphical interface workflow so that user input is not required to populate the one or more corresponding parameter fields within the first graphical interface workflow; and
causing the second default parameters to be stored in the second infusion device to populate one or more corresponding parameter fields within the second graphical interface workflow so that user input is not required to populate the one or more corresponding parameter fields within the second graphical interface workflow.

11. The machine implemented method of claim 10, wherein populating the one or more corresponding parameter fields within the first graphical interface workflow with the first default parameters causes at least one workflow step of the first graphical interface workflow to be pre-completed so that user input is not required for completion of the at least one workflow step of the first graphical interface workflow, and wherein populating the one or more corresponding parameter fields within the second graphical interface workflow with the first default parameters causes at least one workflow step of the second graphical interface workflow to be pre-completed so that user input is not required for completion of the at least one workflow step of the second graphical interface workflow.

12. The machine implemented method of claim 1, wherein the first infusion device and the second infusion device comprise a syringe pump or a large volume pump.

13. A system for programming infusion devices for target controlled infusion, comprising:
a docking station; and
one or more processors configured to:
determine a first infusion device docked with a docking station configured to receive a plurality of infusion devices;
receive, via a first graphical interface workflow displayed on the first infusion device, user input of one or more of patient-specific parameters, drug-related parameters, and a desired target concentration of a therapeutic agent for programming and initiation of a first target controlled infusion (TCI) therapy provided by the first infusion device;
determine when a second infusion device becomes docked with the docking station;
after receiving the patient-specific parameters, the drug-related parameters, or the desired target concentration, and responsive to determining that the first and second infusion devices are simultaneously docked with the docking station, copy a set of parameters to the second infusion device to pre-complete a number of workflow steps of a second graphical interface workflow for programming and initiating a second TCI therapy provided by the second infusion device; and
wherein, after copying the set of parameters to the second infusion device and the second graphical interface workflow is displayed on a display screen of the second infusion device, the number of workflow steps of the second graphical interface workflow are pre-completed and bypassed such that user input is not required for completion of the number of workflow steps of the second graphical interface workflow to program and initiate the second TCI therapy, wherein the number of workflow steps of the second graphical interface workflow would require user input had the set of parameters not been copied.

14. The system of claim 13, wherein the one or more processors are further configured to:
receiving an indication that the second graphical interface workflow was completed on the second infusion device for programming and initiation of a second TCI therapy provided by the second infusion device; and
causing the second infusion device to initiate the second TCI therapy based on the set of parameters copied to the second infusion device.

15. The system of claim 13, wherein the one or more processors are further configured to, after the second infusion device becomes docked with the docking station and before the set of parameters is copied:
cause the first infusion device to display, on a display screen of the first infusion device, a prompt to confirm the copying of the set of parameters,
wherein the set of parameters is copied responsive to receiving a confirmation of the prompt.

16. The system of claim 15, wherein the one or more processors are further configured to:
cause the prompt to be displayed after receiving the patient-specific parameters,
wherein the patient-specific parameters include one or more of an age, weight, sex, height, and a health condition, and
wherein the number of workflow steps that are pre-completed and bypassed on the second graphical interface workflow comprise workflow steps for receiving user input of the patient-specific parameters.

17. The system of claim 13, wherein the one or more processors are further configured to:
receive a request to place the first infusion device in a standby mode;
assign a predetermined color to the received drug type; and
change a current color of at least a portion of the display screen of the first infusion device from a first color to the assigned screen color.

18. The system of claim 13, wherein the one or more processors are further configured to:
determine, while the first TCI therapy is being provided by the first infusion device, that at least one patient-specific parameter of the patient-specific parameters was updated in the second graphical interface workflow of the second infusion device; and
responsive to determining that the at least one patient-specific parameter of the patient-specific parameters was updated in the second graphical interface workflow of the second infusion device, cause the first infusion device to display a prompt for updating the at least one patient-specific parameter in the first graphical interface workflow of the first infusion device to match the at least one patient-specific parameter updated in the second graphical interface workflow of the second infusion device;
receive a confirmation of the prompt; and
responsive to the confirmation, automatically update the at least one patient-specific parameter in the first graphical interface workflow of the first infusion device to match the at least one patient-specific parameter updated in the second graphical interface workflow of the second infusion device.

19. The system of claim 13, wherein the one or more processors are further configured to:
receive, from a server remote from the docking station, a therapy setup library comprising first default parameters for prepopulating a first subset of the drug-related parameters in a respective workflow of a first type of infusion device and second default parameters for prepopulating a second subset of the drug-related parameters in a respective workflow of a second type of the infusion device;
responsive to receiving the therapy setup library:
determine that the first infusion device is of the first type of infusion device and the second infusion device is of the second type of infusion device;
cause the first default parameters to be stored in the first infusion device to populate one or more corresponding parameter fields within the first graphical interface workflow so that user input is not required to populate the one or more corresponding parameter fields within the first graphical interface workflow; and
cause the second default parameters to be stored in the second infusion device to populate one or more corresponding parameter fields within the second graphical interface workflow so that user input is not required to populate the one or more corresponding parameter fields within the second graphical interface workflow.

20. A non-transitory machine readable medium storing instructions thereon that, when executed by one or more processors, cause the one or more processors to perform operations comprising:
determining a first infusion device docked with a docking station configured to receive a plurality of infusion devices;
receiving, via a first graphical interface workflow displayed on the first infusion device, user input of one or more of patient-specific parameters, drug-related parameters, and a desired target concentration of a therapeutic agent for programming and initiation of a first target controlled infusion (TCI) therapy provided by the first infusion device;
determining when a second infusion device becomes docked with the docking station;
after receiving the patient-specific parameters, the drug-related parameters, or the desired target concentration, and responsive to determining that the first and second infusion devices are simultaneously docked with the docking station, copying a set of parameters to the second infusion device to pre-complete a number of workflow steps of a second graphical interface workflow for programming and initiating a second TCI therapy provided by the second infusion device; and
wherein, after copying the set of parameters to the second infusion device and the second graphical interface workflow is displayed on a display screen of the second infusion device, the number of workflow steps of the second graphical interface workflow are pre-completed and bypassed such that user input is not required for completion of the number of workflow steps of the second graphical interface workflow to program and initiate the second TCI therapy, wherein the number of workflow steps of the second graphical interface workflow would require user input had the set of parameters not been copied.
